(19) United States
(12) Patent Application Publication
(10) Pub. No.: US 2020/0222279 A1
(43) Pub. Date: Jul. 16, 2020

(54) WEARABLE DEVICE FOR DELIVERING ELECTRICAL STIMULATION TO THE P6 ACUPUNCTURE POINT

(71) Applicant: ReliefBand Technologies, LLC, Horshan, PA (US)

(72) Inventors: Nicholas Spring, Jenkintown, PA (US); Jacqueline Simon, New Hope, PA (US)

(21) Appl. No.: 16/240,988

(22) Filed: Jan. 7, 2019

Related U.S. Application Data
(60) Provisional application No. 62/614,212, filed on Jan. 5, 2018.

(51) Int. Cl. A61H 39/00; A61N 1/36; A61N 1/04; A61L 31/02

(52) U.S. Cl. CPC A61H 39/002; A61N 1/36034; A61N 1/0476; B22F 3/225; A61H 2201/5043; A61H 2201/1645; A61L 31/022

(57) ABSTRACT

A P6 point electrostimulation device is provided that has a first band, a user interface housing, and a second band. The first band has a free end and is attached to the user interface housing at its other end. The user interface housing has an inner dorsum surface, an outer display surface, a first end that is attached to the first band, a second end that is attached to the second band, user controls, and electronic control circuitry, which includes a power source and pulse generation circuitry. The second band comprises a flexibly rigid curved section which comprises an inside curved surface, an outside curved surface and a pair of electrical conduits and a palmar section which comprises a flat inner surface that has a pair of electrodes, and an outer surface. The flexibly rigid curved section is attached to the user interface housing at one end and to the palmar section at the other end. Each electrical conduit is attached to an electrode at one end and to the electronic control circuitry at the opposite end and the flat inner surface of the palmar section faces the inner dorsum surface of the user interface housing.

### CROSS-REFERENCE TO RELATED APPLICATIONS

The present application claims priority to U.S. Provisional Patent Application Ser. No. 62/614,212, filed on Jan. 5, 2018”, the disclosure of which is hereby incorporated in its entirety at least by reference.

### FIELD OF THE INVENTION

The devices and methods described below relate to the fields of wearable devices for delivering noninvasive electrical stimulation to the P6 acupuncture point and methods of using the same.

### BACKGROUND OF THE INVENTION

Bertolucci, Nausea Control Device, U.S. Pat. No. 4,981,146, Jan. 1, 1991, which is incorporated herein by reference, describes a nausea control device (the Bertolucci device) in the form of a watch-like housing attachable to the human wrist by an adjustable attachment band. The device uses non-invasive nerve stimulation whereby electricity is passed through two electrodes to stimulate nerves located on the ventral side of the wrist (this anatomical position is sometimes referred to as the palmar side of the wrist). The ventral site of the wrist is referred to as the P6 point, pericardium 6 point, or master point of the pericardium meridian (sometimes referred to as the vascular meridian). Bertolucci describes the device as providing a non-chemical, non-invasive, painless and inexpensive method of alleviating nausea that is portable, self-contained and convenient to the patient. As described in Bertolucci, the electrical pulse repetition rate of approximately 70 pulses per second and a pulse width of 80 microseconds has been found to provide effective relief of nausea in a patient. Bertolucci discloses an electrical pulse pattern as comprising about 350 microsecond pulse width at about 31 pulses per second at power levels of about 10-35 milli-amps peak pulse height.

A wide range of pulse patterns may be used in non-invasive nerve stimulation devices. Nausea control devices similar to those described in U.S. Pat. No. 4,981,146 have been sold to treat nausea caused by chemotherapy, motion sickness and morning sickness associated with pregnancy and as an adjunct to antiemetic therapy in reducing post-operative nausea. U.S. Pat. Nos. 6,567,695 and 7,127,288, which are each incorporated herein by reference, disclose methods and apparatus for low power, regulated output in battery powered electrotherapy devices. U.S. Pat. Nos. 6,076,018 and 7,171,266, which are each incorporated herein by reference, disclose electrotherapy devices with stimulation electrode assemblies. U.S. Pat. Nos. 6,735,480 and 7,983,761, which are each incorporated herein by reference, disclose electrotherapy devices with electrode design features. Similar devices have been disclosed for use in treating dysmenorrhea and menstrual cramps (U.S. Pat. Nos. 6,282,443 and 6,718,202, which are each incorporated herein by reference), for use in moderating blood pressure (U.S. Pat. Nos. 6,178,352 and 6,658,298, which are each incorporated herein by reference) and for reducing gag reflex (U.S. Pat. No. 6,192,889, which is incorporated herein by reference).

### SUMMARY OF THE INVENTION

A P6 point electrostimulation device is provided that includes a first band, a user interface housing, and a second band. The simple band includes a first end that is a free end and a second end that is attached to the user interface housing. The user interface housing includes an inner dorsum surface, an outer display surface, a first end that is attached to the first band, a second end that is attached to the second band, user controls, and electronic control circuitry. The electronic control circuitry includes a power source and pulse generation circuitry. The second band includes a flexibly rigid curved section and a palmar section. The flexibly rigid curved section is attached to the user interface housing at one end and to the palmar section at the other end. The flexibly rigid curved section includes an inside curved surface, an outside curved surface and a pair of electrical conduits. The palmar section comprises a flat inner surface that comprises a pair of electrodes and outer surface. Each electrical conduit is attached to an electrode at one end and to the electronic control circuitry at the opposite end. The flat inner surface of the palmar section faces the inner dorsum surface of the user interface housing.

### BRIEF DESCRIPTION OF THE FIGURES

FIGS. 1A, 1B, and 1C show side and perspective views of an embodiment of the P6 electro-stimulation device;

FIG. 2 shows the same side view of an embodiment of the P6 electro-stimulation device as FIG. 1. FIG. 2 identifies various measurements;

FIGS. 3A and 3B show views of a J-band. FIG. 3A shows a view of an assembled J-band and FIG. 3B shows an exploded view of a J-band;

FIG. 4A shows a J-band without an attached clasp assembly;

FIG. 4B shows a J-band with an attached clasp assembly;

FIG. 4C shows an exploded view of a J-band;

FIG. 5 shows attachment of an electrode to an electrical conduit;

FIG. 6 shows a diagram of the electronic control circuitry;

### DETAILED DESCRIPTION OF PREFERRED EMBODIMENTS

Provided herein is a non-invasive device indicated for over-the-counter use in the relief of nausea, retching and vomiting due to motion sickness, pregnancy, chemotherapy as well as for use as an adjunct to antiemetics for the relief of post-operative nausea, retching and vomiting. Additional uses for the device may include treating dysmenorrhea and menstrual cramps, reduction of gag reflex, and modulation of blood pressure. In addition, as described in patent applications, entitled additional uses may include treatment of alcohol hangover, treatment of vertigo and prevention and treatment of virtual reality sickness. The device is contained within a wristband, and provides relief through electrical stimulation of the median nerves in the user's wrist. Specifically, the device deliverers electro stimulation to the acupuncture point on the ventral or palmar side of an individual wrist known as the P6 point, pericardium 6 point, or master point of the pericardium meridian (sometimes referred to as the vascular meridian).

The P6 electrostimulation device provided herein is a battery-powered therapeutic Class II medical device that operates by transcutaneous electrical stimulation of the P6 acupressure point on the ventral, or inside, surface of the wrist, also referred to as the palmar side of the wrist, which is on the same side as the palm of the hand. It can be worn and used on either or both wrists. The P6 electrostimulation device provided herein has a user interface (UI) housing which comprises user controls, indicator output such as LEDs or a display, and electronic control circuitry including a power source.

The disclosed P6 electrostimulation device provided herein includes components which allow for ease of attachment and placement on a user's wrist. Two bands or straps are provided that are attached to the UI housing at opposite ends from each other. A first band may be a simple strap or band feature which is flexible and configured to wrap around a portion of a user's wrist. A second band, referred to in an exemplary embodiment as a J-band, has a clasp mechanism distal to the end of the J-band that is attached to the UI housing. The J-band further comprises two electrodes on its inside surface at the end where the clasp mechanism is present on the outside surface. The J-band further comprises an electrical conduit connecting the electrodes to the electrical circuit controls in the UI housing. The portion of the J-band between the end attached to the UI housing and the end that comprises the electrodes and clasp mechanism is flexibly rigid in a preform curved arc shape that maintains a curved preform in the unloaded state. The design provides that the UI housing is attached to the flexibly rigid, preformed curved section, with the electrodes facing the surface of the UI housing configured to be in contact with the wrist when worn by the user. A second band, referred to as a simple band, has a free end distal to the end of the simple band that is attached to the UI housing. The P6 electrostimulation device provided herein is securely positioned on the user's wrist by engaging the free end of the simple band with the clasp mechanism on the J-band. When the two bands are adjusted relative to each other to achieve a secure, comfortable fit positioned for therapy, the clasp mechanism locks the bands in place relative to each other.

When worn by the user, the P6 electrostimulation device provided herein is positioned for therapy with such that two electrodes are located approximately 2 fingers breadth above the distal skin crease of the wrist joint, between the tendons of the palmaris longus and flexor carpi radialis muscles. When worn, the electrodes are in contact with the P6 point at the ventral or palmar side of the wrist. The UI housing is worn in the approximate location of a traditional watch face on the dorsal or dorsum side of the wrist, that is the same side as the back of the hand. The UI housing is in contact with the dorsal or dorsum side of the wrist. The UI allows the user to turn the unit on and off, set and verify the intensity level of stimulation, and determine the level of charge on the rechargeable battery.

The P6 electrostimulation device provided herein may be put on by the user quickly and easily with one hand, and adjusted for proper electrode location and band tightness in a fashion that promotes confidence and repeatability of effective therapy. The essentially J-shaped configuration of the J-band, with its flexibly rigid preform curved section, attached to the UI housing facilitates the ease of putting on the device with one hand. The essentially J-shaped J-band plus UI housing allows the device to be hung on the wrist with the electrodes positioned properly. When placed on the user's wrist, the electrodes of the J-band are positioned at and in contact with the P6 point on the wrist and the inner surface of the UI housing is positioned at and in contact with the dorsal or dorsum side of the wrist, i.e. the side of the wrist adjacent to the back of the hand. Once hung on the wrist of one hand, the user can use their other hand to insert the free end of the simple band into the clasp mechanism on the J-band. The curvature and flexibly rigid preform of the J-band holds the J-shaped J-band and UI housing portion in place on the wrist to allow the user to place the simple band into a clasp located on the J-band. The clasp mechanism has a cam and cinch bar which allows for the bands to be firmly adjusted and locked at any position along the length of the simple band. The clasp design that allows for precise adjustment of the simple band, thereby providing ease of positioning and closure with the electrodes in the proper position for use of the device.

The simple band and J-band may be locked to firmly secure and position the device to accommodate the circumference of the user's wrist. Thus, the design of the P6 electrostimulation device provided herein enables the user to quickly and easily put the device on with one hand, and adjust it so that the electrodes are positioned at the proper location for effective therapy.

The dimensions of the P6 electrostimulation device are provided to accommodate wrist sizes from having a wrist circumference from about 13.7 cm to about 19.3 cm. This range of wrist sizes represents the range from approximately the 5^{th} percentile of adult women's wrist sizes to the 95^{th} percentile of adult men's wrist sizes.

The User Interface allows for the user to turn the device on, set the intensity of stimulation and verify adequate battery charge remaining in just a few seconds and with confidence. In some embodiments, the P6 electrostimulation device provided herein may be comfortably and effectively used for two weeks continuously, interrupted by charging intervals and gel applications as described in the user instructions for use (IFU). In some embodiments, the P6 electrostimulation device provided herein will supply 15-45 hours (+/−15%) of continuous therapy when the intensity is set to the middle level and the battery has been fully charged. In some embodiments, the P6 electrostimulation device provided herein may further comprise conductivity gel for use as described in the IFU, and a USB charging cable. In some embodiments, the P6 electrostimulation device provided herein may be able to be periodically cleaned by wiping down with a damp cloth, or a cloth with mild detergent or isopropyl alcohol.

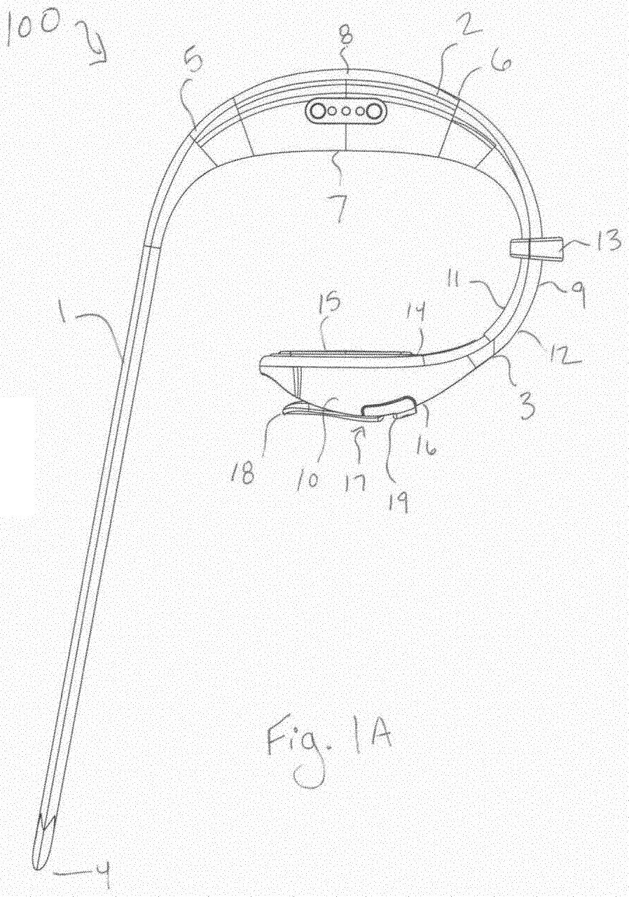

FIG. 1A shows a side view of an embodiment of the P6 electro-stimulation device 100. FIGS. 1B and 1C show perspective views of the device 100, further illustrating the features described below. Three main sections of the device 100 are shown: a simple band 1, a user interface housing 2 and a J-band 3. The terms simple band and J-band are used for the sake of convenience and are not limited to simple and/or J-band components. For example, the simple band 1 may be a first band which forms a first portion of the device 100 and the J-band 3 a second band which forms a second portion of the device 100. Together with the interface housing 2, these first and second bands form an enclosed space for receiving the wrist of the user.

The simple band 1 has a free end 4. The simple band 1 and the user interface housing 2 are attached to each other at the simple band-user interface housing attachment point 5. The user interface housing 2 is attached to the J-band 3 at the J-band-user interface housing attachment point 6. The user interface housing 2 has an inner dorsum surface 7 and an outer display surface 8 and preferably a slight curve for better fit when in contact with the dorsum side of the user's wrist. The J-band 3 has a flexibly rigid curved section 9 and a palmar section 10. The flexibly rigid curved section 9 has inside curved surface 11, an outside curved surface 12 and a strap loop 13. The palmar section 10 comprises the flat inner surface 14 that comprises a pair of electrodes 15 separated by a gap (shown in FIG. 1B) and outer surface 16 comprising a clasp assembly 17 that comprises a cam 18 and a cinch bar 19. Without a load on the flexibly rigid curved section 9, the flat inner surface 14 of the palmar section 10 of the J-band 3 faces the inner dorsum surface 7 of the user interface housing 2.

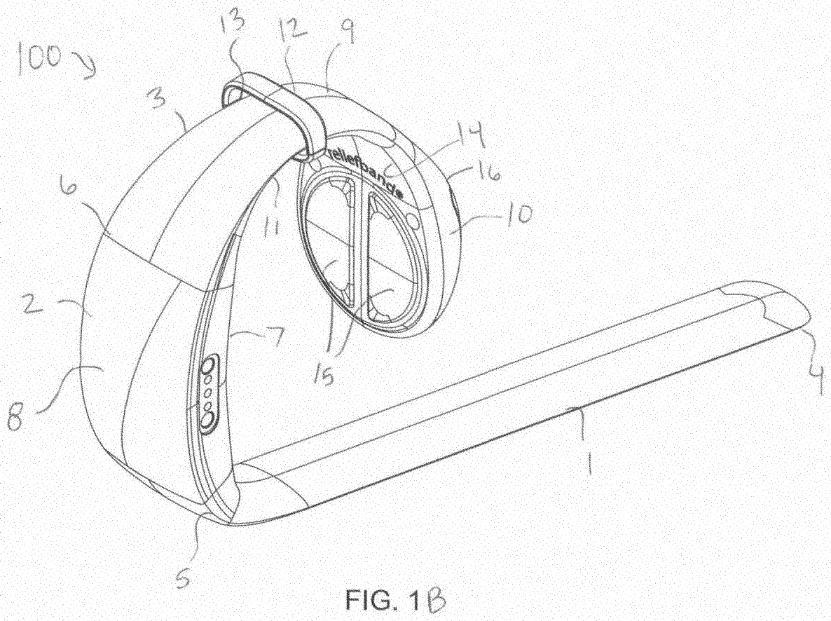

FIG. 2 shows the same side view of an embodiment of the P6 electro-stimulation device 100. In FIG. 2, the dashed line 20 represents a straight line that is perpendicular to the flat inner surface 14 of the palmar section 10 of the J-band 3 arising from a point in the gap between the pair of electrodes 15. The center point 21 of dashed line 20 is shown as “+”. The dashed line 22 represents a straight line extending from center point 21 to the point on the inside curved surface 11 of the flexibly rigid curved section 9 of J-band 3 furthest from center point 21. The dashed line 23 represents a straight line extending from center point 21 to the point on the inside curved surface 11 of the flexibly rigid curved section 9 of J-band 3 closest from center point 21.

In some embodiments, the distance corresponding to dashed line 20 is between 30 cm and 40 cm when without load. In some embodiments, the distance corresponding to dashed line 20 is between 30.5 cm and 39.5 cm when without load. In some embodiments, the distance corresponding to dashed line 20 is between 31 cm and 39 cm when without load. In some embodiments, the distance corresponding to dashed line 20 is between 31.5 cm and 38.5 cm when without load. In some embodiments, the distance corresponding to dashed line 20 is between 32 cm and 38 cm when without load. In some embodiments, the distance corresponding to dashed line 20 is between 32.5 cm and 37.5 cm when without load. In some embodiments, the distance corresponding to dashed line 20 is between 33 cm and 37 cm when without load.

In some embodiments, the distance corresponding to dashed line 20 is between 33.5 cm and 36.5 cm when without load. In some embodiments, the distance corresponding to dashed line 20 is between 34 cm and 36 cm when without load. In some embodiments, the distance corresponding to dashed line 20 is 35 cm+/−1 cm when without load.

When flexed outward to increase the distance, the flexible rigidity provides that the distance corresponding to dashed line 20 may be between 45 cm and 55 cm, in some embodiments. When flexed outward to increase the distance, the flexible rigidity provides that the distance corresponding to dashed line 20 may be between 45.5 cm and 54.5 cm. When flexed outward to increase the distance, the flexible rigidity provides that the distance corresponding to dashed line 20 may be between 46 cm and 54 cm. When flexed outward to increase the distance, the flexible rigidity provides that the distance corresponding to dashed line 20 may be between 46.5 cm and 53.5 cm. When flexed outward to increase the distance, the flexible rigidity provides that the distance corresponding to dashed line 20 may be between 47 cm and 53 cm. When flexed outward to increase the distance, the flexible rigidity provides that the distance corresponding to dashed line 20 may be between 47.5 cm and 52.5 cm. When flexed outward to increase the distance, the flexible rigidity provides that the distance corresponding to dashed line 20 may be between 48 cm and 52 cm. When flexed outward to increase the distance, the flexible rigidity provides that the distance corresponding to dashed line 20 may be between 48.5 cm and 51.5 cm. When flexed outward to increase the distance, the flexible rigidity provides that the distance corresponding to dashed line 20 may be between 49 cm and 51 cm. When flexed outward to increase the distance, the flexible rigidity provides that the distance corresponding to dashed line 20 may be 50 cm+/−1 cm.

When flexed inward to decrease the distance, the flexible rigidity provides that the distance corresponding to dashed line 20 may be between 16 cm and 24 cm, in some embodiments. When flexed inward to decrease the distance, the flexible rigidity provides that the distance corresponding to dashed line 20 may be between 16.5 cm and 23.5 cm. When flexed inward to decrease the distance, the flexible rigidity provides that the distance corresponding to dashed line 20 may be between 17 cm and 23 cm. When flexed inward to decrease the distance, the flexible rigidity provides that the distance corresponding to dashed line 20 may be between 17.5 cm and 22.5 cm. When flexed inward to decrease the distance, the flexible rigidity provides that the distance corresponding to dashed line 20 may be between 18 cm and 22 cm. When flexed inward to decrease the distance, the flexible rigidity provides that the distance corresponding to dashed line 20 may be between 18.5 cm and 21.5 cm. When flexed inward to decrease the distance, the flexible rigidity provides that the distance corresponding to dashed line 20 may be between 19 cm and 21 cm. When flexed inward to decrease the distance, the flexible rigidity provides that the distance corresponding to dashed line 20 may be 20 cm+/−1 cm.

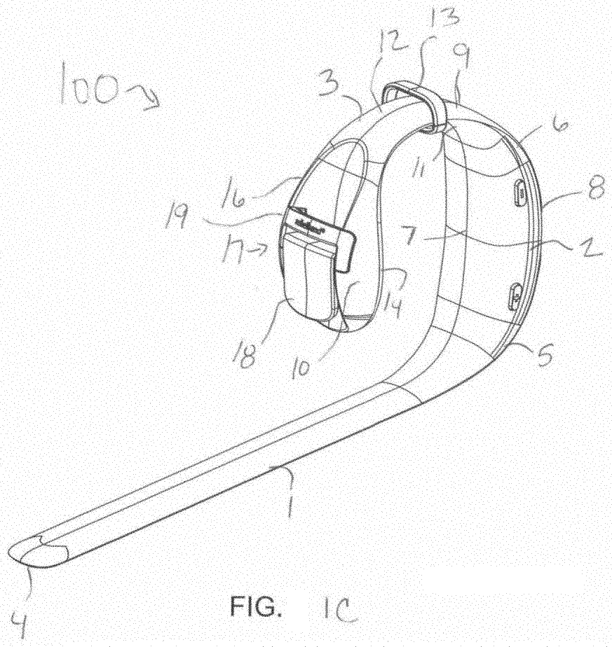

FIG. 3A shows an embodiment of the assembled J-band 3 of the exemplary device 100. The J-band 3 has the flexibly rigid curved section 9 and the palmar section 10. The flexibly rigid curved section 9 has the inside curved surface 11 and the outside curved surface 12. The curved section 9 is shaped and configured to wrap around the side of a person's wrist such that the palmar section 10 may be positioned adjacent to the inside of the wrist. The palmar section 10 has the flat inner surface 14 that comprises the pair of electrodes 15 separated by a gap 24. The electrodes 15 are configured to contact the inside of the person's wrist.

FIG. 3B shows an exploded view of the exemplary J-band 3. In an exemplary embodiment, the J-band 3 includes a skeleton section 25, a palmar frame 27, and a backbone 28. The skeleton section 25 comprises the outer surface 16 of the palmar section. The palmar frame 27 and the backbone 28 of the flexibly rigid curved section 9 provide the preform curved shape and flexible rigidity of the J-band 3. The backbone 28 is connected to the palmar frame 27 at one end of the backbone 28 and comprises a conduit connection frame 29 at the other end of the backbone 28. A pair of electrodes 15 are attached to electrode frames 30. A pair of electrical conduits 31 are provided. Electrodes 15 are separately attached to electrical conduits 31 at one end of the electrical conduits 31 and the opposite end of the electrical conduits 31 are positioned within the conduit connection frame 29.

The pair of electrodes 15, which are attached to electrode frames 30, and the pair of electrical conduits 31, which are attached to the backbone 28 at the conduit connection frame 29 are positioned within a J-band primary overmold 32. The electrodes 15 are seated in electrode openings 33 of the J-band primary overmold 32 and separated by the gap 24. The electrical conduits 31 and backbone 28 are positioned within the J-band primary overmold 32. The J-band primary overmold 32 comprises the inside curved surface 11 and an intermediate layer 34. The conduit connection frame 29 and the end of the electrical conduits 31 are positioned at an end of the J-band primary overmold 32 in a conduit connection port 35. Ends of the electrical conduits 31 positioned in the conduit connection port 35 are held in place in the port by a pair fastening blocks 36. A back section overmold 37 is attached over the intermediate layer 34 and forms the outside curved surface 12.

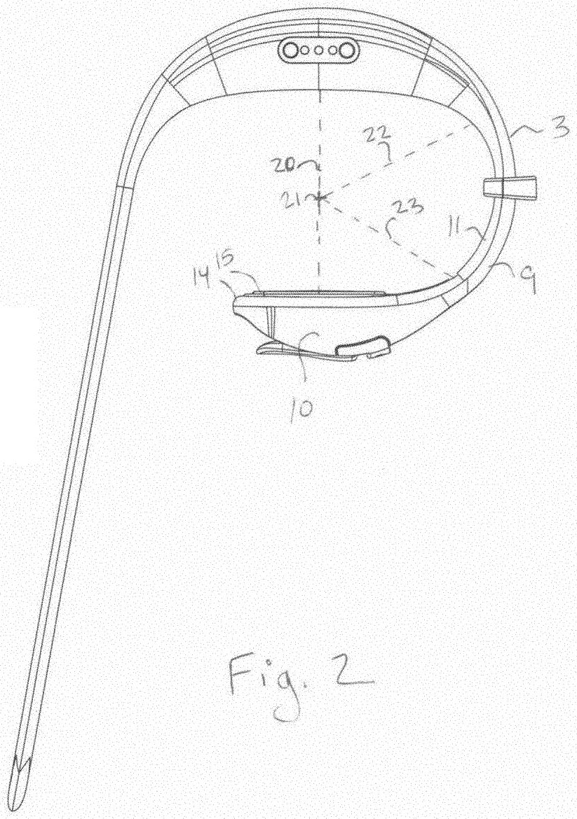

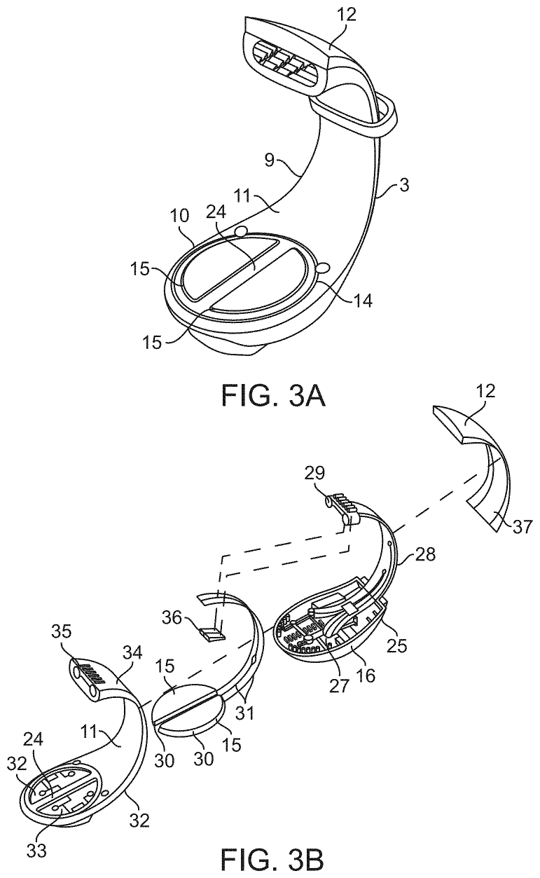

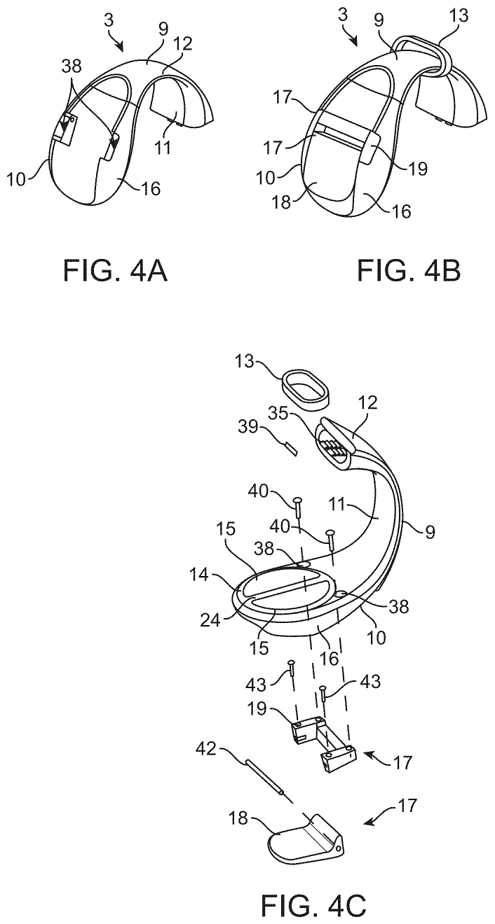

FIG. 4A shows the J-band 3 without an attached clasp assembly. The J-band 3 has a flexibly rigid curved section 9 and a palmar section 10. The flexibly rigid curved section 9 has inside curved surface 11 and an outside curved surface 12. The palmar section 10 has an outer surface 16 comprising attachment sites 38 where a clasp assembly (not shown) may be mounted.

FIG. 4B shows the J-band 3 with attached clasp assembly 17. The J-band 3 has the flexibly rigid curved section 9, the palmar section 10 and a strap loop 13. The palmar section 10 has the outer surface 16 which comprises the clasp assembly 17. In an exemplary embodiment, the clasp assembly 17 comprises a cam 18 and a cinch bar 19. The cam 18 and cinch bar 19 form space for capturing the end 4 of the simple band 1 and thereby attaching the simple band 1 to the J-band 3.

FIG. 4C shows an exploded view of an embodiment of the J-band 3. The J-band 3 has the flexibly rigid curved section 9, the palmar section 10 and the strap loop 13. The flexibly rigid curved section 9 has the inside curved surface 11 and the outside curved surface 12. The palmar section 10 has the flat inner surface 14 that comprises the pair of electrodes 15 separated by the gap 24 and outer surface 16 comprising the clasp assembly 17. In an exemplary embodiment, an O-ring 39 is provided at the conduit connection port 35. A pair of clasp attachment screws 40 are provided for attaching the cinch bar 19 to the palmar section 10 at attachment sites 38. The cam 18 is fastened to the cinch bar 19 by a metal pin 42 that is held in place by a pair of metal dowels 43.

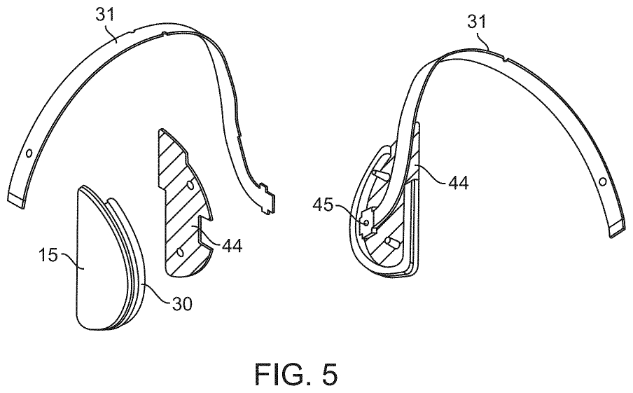

FIG. 5 shows attachment of an electrode 15 to an electrical conduit 31. The electrode 15 is attached to an electrode frame 30 and an electrode insulator 44. The electrical conduit 31 is attached to the electrode 15 by an electrode fastener tab 45. The electrical conduit 31 is preferably formed in a curved shape that matches the flexibly rigid curved section 9 such that the electrical conduit 31 may traverse from the electrodes 15 to the user interface housing 2. The user interface housing 2 preferably includes electronics which connect to the electrical conduit 31.

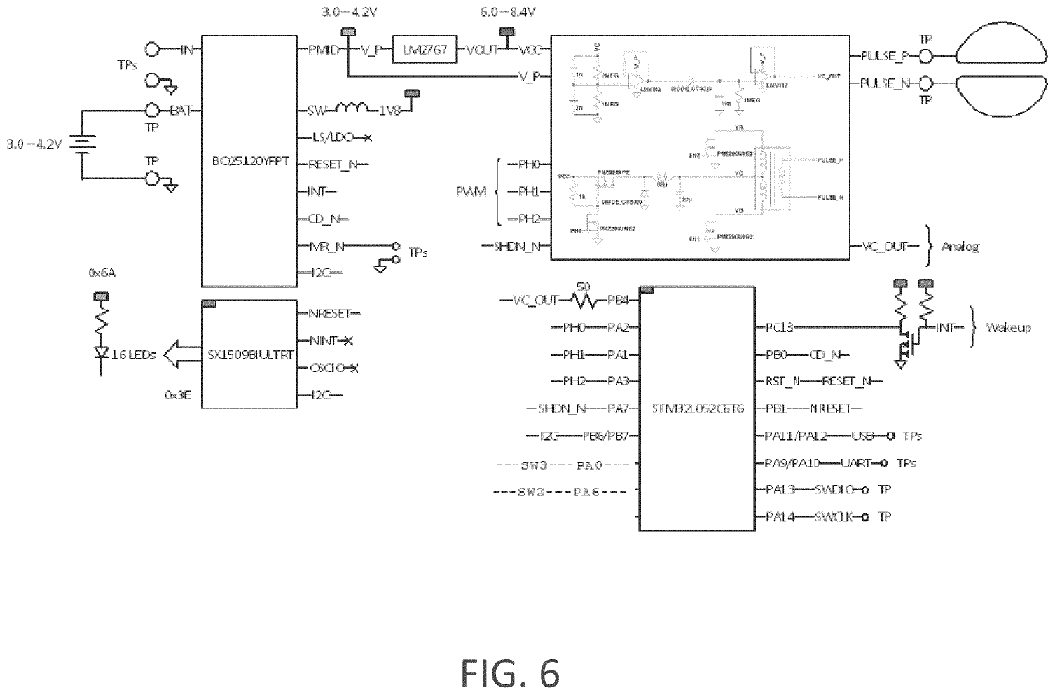

FIG. 6 shows a diagram of an exemplary electronic control circuitry which may be used in conjunction with the device 100.

It should be understood that these circuit diagrams are exemplary, and that variations or alternatives may be used. Other electrical design and pulse generation provided by the P6 electrostimulation device provided herein may correspond to any of the electrical designs and pulse generation specifications described any of U.S. Pat. No. 4,981,146, 6.076,018, 6,567,695, 6,735,480, 7,127,288, 7,171,266, 7,171,276 or 7,983,761. The electronic circuitry of the device may be configured to provide electrical stimulation to a person's wrist while utilizing the J-shaped construction of a J-band 3 as described herein. The construction allows the user interface housing 2 to be conveniently positioned on the top of the person's wrist while also properly positioning the electrodes 15 on the opposite underside of the wrist. The simple band 1 compliments the J-band 3 to form an enclosure that maintains the device 100 on the person.

In some embodiments, the P6 electrostimulation device provided herein the pulse generation (output) of the device is as follows. Waveform may be asymmetrical balanced and biphasic. In some embodiments, ten (10) intensity levels (defined by the maximum current amplitude measured at the peak of the stimulus pulse when unit is driving a 500 ohm purely resistive load) will be available (all expressed as “setting level=output in milliamperes”) are provided. In some embodiments, the ten intensity settings may be as follows: 1=8, 2=11, 3=14, 4=17, 5=20, 6=24, 7=28, 8=32, 9=36, 10=40, at tolerances of ±15%. In some embodiments, amplitude to be nominally constant current (+/−20% of output into 500-ohm load), tested at 200 ohms and 1000-ohm loads. In some embodiments, amplitude may be automatically modulated using a 1 second ramp up to setting, 2 seconds constant at setting, 1 second ramp down to zero or lowest possible value, repeating continuously. In some embodiments, pulse width may be 350 μseconds+/−10%. In some embodiments, pulse frequency may be 31 Hz (32 msec period between alternating monophasic pulses)+/−10%. In some embodiments, pulse waveform may be such that the ratio of: [pulse amplitude at 50+/−2 μseconds after the start of the pulse] to [peak pulse amplitude at start of pulse] may be equal to 0.51+/−12%. In some embodiments, waveform will be such that the ratio of: [pulse amplitude at 100+/−4 μseconds after the start of the pulse] to [peak pulse amplitude at start of pulse] will be equal to 0.26+/−20%. In some embodiments, the device may incorporate an output regulation feature such that the stimulation output amplitude does not vary more than 20% from the nominal as the battery charge decreases while in use. In some embodiments, the device may incorporate an automatic shut off feature such that when output regulation cannot be maintained for any setting, the device will shut off. In some embodiments, battery capacity and performance will deliver a minimum of 16 hours of continuous therapy from a full charge at intensity level 5 (which is half of the maximum output, or 20 mA output level) when tested using a 500 ohm purely resistive load.

In some embodiments, the P6 electrostimulation device is provided herein incorporates the following features with respect to battery and charging. In some embodiments, the battery may be a rechargeable type with a target life of 500 discharge cycles. In some embodiments, the time to charge the rechargeable battery from 80% depleted is be no greater than 45 minutes, and less than 75 minutes from fully discharged with the device not delivering therapy. In some embodiments, charging may be accomplished in any operational state of the device and may commence upon connection of the charging cable to both an appropriate power source (for example a USB wall charger) and the device. In some embodiments, a written description of the battery monitoring system is provided. In some embodiments, the device may use a charging adapter cable with USB-A on the adapter side and a magnetically attached connector on the device side.

In some embodiments, the P6 electrostimulation device provided herein incorporates software with the following features. For example, the device software may establish the specified relationships between control inputs to device outputs and monitor the battery and charging behavior and status and act as needed to reduce risk of failure or harm.

In some embodiments, the P6 electrostimulation device provided herein incorporates the following features with respect to the band/strap and electrodes. In some embodiments, the band is designed to fit the range of wrist circumference sizes from the 5th percentile female to the 95th percentile male. “Fit” is meant to refer to the comfort and wear ability with the electrodes simultaneously held against the user's skin with adequate force to effect therapy, and the electrodes' location optimized while maintaining good readability of the information on the UI.

The P6 electrostimulation device provided herein comprises a band secured together by a closure. One side of the band, referred to in an exemplary embodiment as the “J-band,” has increased rigidity and maintains a preform in the unloaded state to allow the user to “hang” the device on the wrist to facilitate one-handed application. The electrodes are preferably made from 316L Stainless Steel, preferably 316L Stainless Steel manufactured by metal injection molding technology. In preferred embodiments, the electrical leads to the electrodes and the rest of the J-band and band attachment must be able to withstand 5,000 flexes from free state to 17 mm radius of curvature on the flex side, and to 25 mm radius of curvature on the preform side.

The free side of the strap, i.e. the side with no electrodes, referred to as the simple band, preferably can withstand 5,000 twist cycles, applied at the terminus, through 90-degrees each way and back to free state. The semi-rigid side of the strap, the J-band, preferably can withstand 5,000 twist cycles, applied at the terminus, through 30-degrees each way and back to free state. Each side of the strap, i.e. the J-band and the simple band, preferably can withstand a 50 Newton in-line pull force with no damage. This force may be applied tangentially using a mandrel for the preformed side, the J-band. When the strap closure, which is a clasp located on the J-band, is engaged, the strap will remain retained with a pull force of 25 N.

In a preferred embodiment, the P6 electrostimulation device comprises a J-band, a user interface housing and a simple band. One end of the J-band is attached to the user interface housing and the other end of the J-band comprises a pair of D shaped electrodes on the inner side of the J-band and a clasp on the outer side of the J-band. One end of the simple band is attached to the user interface housing and the other end of the simple band is configured to engage with the clasp on the outer side of the J-band. The J-band, which comprises one end that attached to the user interface housing, one end that comprises a pair of 1) shaped electrodes on the inner side of the J-band and a clasp on the outer side of the J-band, is flexibly rigid and maintains a preform shape. In preferred embodiments, an electrode connector guide made of flexibly rigid material is provided which gives the J-band its flexible rigidity and preformed shape. An electrical connector is provided, which connects the electrodes to the electronic control circuitry in the user interface housing.

In a preferred embodiment, electrodes used in the P6 electrostimulation devices provided herein are made of 316L stainless steel, preferably manufactured using metal injection molding technology. Such electrodes have been found to provide more comfort and less incidence of painful incidents in use compared to devices made using electrodes used in previous devices that were made from different material stamped from rolled sheets of metal. The problems associated with the discomfort and occasional pain experience using the electrodes of previous P6 electrostimulation devices result in the need to reduce the power output when using the device to minimize discomfort and incidence of pain. Providing electrodes made of 316L stainless steel manufactured using metal injection molding technology in P6 electrostimulation devices allows for the use of such devices set to deliver pulses at higher power output because of the reduced incidences of discomfort and pain. P6 electrostimulation devices that comprise electrodes made of 316L stainless steel manufactured using metal injection molding technology thus may be more effective compared to previous comparable devices.

Metal injection molding technology provides processes for injection molding articles from metal powders including 316L stainless steel powder. Examples of metal injection molding technology are described in U.S. Pat. Nos. 4,113,480, 4,227,926, 4,305,757, 4,404,166, 4,478,790, 5,059,388, 5,985,208, 6,056,915 and 6,846,862, and U.S. Patent Application Nos. US20080015683, US20060276875, which are incorporated herein by reference.

In a preferred embodiment, the P6 electrostimulation devices comprises a pair of metal electrodes made of 316L stainless steel manufactured using metal injection molding technology that are substantially D-shaped, i.e. having a straight edge and an arcuate edge. The electrodes made of 316L stainless steel manufactured using metal injection molding technology are arranged such that when the device is properly worn by a user and the electrodes are in contact with the P6 point on a user's wrist and the straight edges of each are opposing each other, i.e. arranged with the straight edges facing each other in apposition. The straight edges are thus parallel to each other. The electrodes are arranged on the device such that when the device is properly worn by a user and the electrodes are in contact with the P6 point on a user's wrist the electrodes are perpendicular with the direction of the users arm between the wrist and elbow. The straight edges have a length of about 1 inch, and the arcuate edges having an inner radius of about 0.5 inches. In a preferred embodiment, D shaped electrode is about 22.00 to about 25.50 mm and the arcuate edges having an inner radius of about 10.50 to about 13.00 mm. In a preferred embodiment, each D shaped electrode has a thickness of about 0.50 to about 1.75 mm. In a preferred embodiment, each D shaped electrode is about 23.00 to about 24.50 mm and the arcuate edges having an inner radius of about 11.00 to about 12.70 mm. In a preferred embodiment, each D shaped electrode has a thickness of about 0.80 to about 1.40 mm. In a preferred embodiment, each D shaped electrode is about 23.84+/−0.10 mm and the arcuate edges having an inner radius of about 11.83+/−0.10 mm. In a preferred embodiment, each D shaped electrode has a thickness of about 1.08+/−0.10 mm.

The electrodes are arranged on the device such that the straight edges of the electrodes face each other in apposition and are separated by a gap. In some embodiments, the gap is 0.05 to 0.5 inches or 1-15 mm. In some embodiments, the gap is 0.1 to 0.25 inches or 2-7.5 mm. In some embodiments, the gap is 3-5 mm. In some embodiments, the gap is 0.14 inches.

In a preferred embodiment, the electrodes are connected to the electrical conduits that supply electrical output at a position on or immediately adjacent to, i.e. within 5 mm, preferably within 3 mm of the center point of the arcuate edge.

The embodiments described herein are exemplary and that other configurations are possible. The disclosed embodiments are not limited by the claims which follow.

1. A P6 point electrostimulation device comprising:
a first band,
a user interface housing, and
a second
wherein
the first band comprises a first end that is a free end and a second end that is attached to the user interface housing,
the user interface housing comprises an inner dorsum surface, an outer display surface, a first end that is attached to the first band, a second end that is attached to the second band, user controls, and electronic control circuitry, the electronic control circuitry comprising a power source and pulse generation circuitry,
the second comprises a flexibly rigid curved section and a palmar section, the flexibly rigid curved section is attached to the user interface housing at one end and the palmar section at the other end, the flexibly rigid curved section comprising an inside curved surface, an outside curved surface and a pair of electrical conduits, the palmar section comprising a flat inner surface that comprises a pair of electrodes and an outer surface, each electrical conduit is attached to an electrode at one end and to the electronic control circuitry at the opposite end, and the flat inner surface of the palmar section faces the inner dorsum surface of the user interface housing.

2. The P6 point electrostimulation device of claim 1, wherein
the pulse generation circuitry generates an electric pulse having a waveform that is asymmetrical balanced and biphasic, ten intensity level settings ranging from <1 to 40 mA controllable by the user controls, amplitude that is nominally constant current and that is automatically modulated using a 1 second ramp up to setting, 2 seconds constant at setting, 1 second ramp down to zero or lowest possible value, repeating continuously, pulse width is 350 μseconds+/−10%, pulse frequency is 31 Hz 32 msec period between alternating monophasic pulses+/−10%, pulse waveform having a ratio of: [pulse amplitude at 50+/−2 μseconds after the start of the pulse] to [peak pulse amplitude at start of pulse] equal to 0.51+/−12%, and, waveform with a ratio of: [pulse amplitude at 100+/−4 μseconds after the start of the pulse] to [peak pulse amplitude at start of pulse] equal to 0.26+/−20%.

3. The P6 point electrostimulation device of claim 2, wherein the ten intensity settings are 1=8, 2=11, 3=14, 4=17, 5=20, 6=24, 7=28, 8=32, 9=36, 10=40, at tolerances of ±15%.

4. The P6 point electrostimulation device of claim 1, wherein the electrodes are each D shaped comprising a straight edge and an arcuate edge and are arranged on the flat inner surface with the straight edges of the electrodes parallel to each other, are separated by a gap.

5. The P6 point electrostimulation device of claim 1, wherein the electrodes are made of 316L stainless steel.

6. The P6 point electrostimulation device of claim 1, wherein the electrodes are made of 316L stainless steel manufactured by metal injection molding.

7. The P6 point electrostimulation device of claim 1, wherein the electrodes are attached to the electrical conduit at a point within 3-5 mm or less of the center of the arcuate edge of the electrode.

8. The P6 point electrostimulation device of claim 1, wherein the flat inner surface of the palmar section faces the inner dorsum surface of the user interface housing and separated at a distance of between 30 cm and 40 cm, between 30.5 cm and 39.5 cm, between 31 cm and 39 cm, between 31.5 cm and 38.5 cm, between 32 cm and 38 cm, between 32.5 cm and 37.5 cm, between 33 cm and 37 cm, between 34 cm and 36 cm or 35 cm.

9. The P6 point electrostimulation device of claim 1, further comprising a clasp assembly on the outer surface of the palmar section that comprises a cam and a cinch bar for receiving the free end of the first band.
